(19) United States
(12) Patent Application Publication
(10) Pub. No.: US 2018/0011089 A1
(43) Pub. Date: Jan. 11, 2018

(54) WEARABLE WITH TEST STRIPS AND OPTICAL READER FOR SKIN

(71) Applicant: KONINKLIJKE PHILIPS N.V., EINDHOVEN (NL)

(72) Inventor: John Cronin, Bonita Springs, FL (US)

(73) Assignee: KONINKLIJKE PHILIPS N.V., EINDHOVEN (NL)

(21) Appl. No.: 15/545,761

(22) PCT Filed: Feb. 1, 2016
(86) PCT No.: PCT/EP2016/052012

§ 371 Date: Jul. 24, 2017

Related U.S. Application Data
(60) Provisional application No. 62/110,967, filed on Feb. 2, 2015.

(30) Foreign Application Priority Data
Jun. 30, 2015 (EP) 15174547.8

(51) Int. Cl. G01N 33/543; A61B 5/1455; A61B 5/00; G01N 21/78; A61B 5/145; A61B 5/20; G01N 21/77

(52) U.S. Cl. CPC G01N 33/54373; A61B 5/14517; A61B 5/1455; A61B 5/207; A61B 5/4277; G01N 21/78; G06T 7/90; G01N 2021/7759; G01N 2201/12; G06T 2207/10024; G06T 2207/30072; A61B 2562/0295

(57) ABSTRACT

The present invention generally relates to a wearable electronic device that collects information about test sample using an optical sensor. The wearable device photographs and analyzes one or more organic samples placed on one or more immunoassay regions of a test strip in order to reveal information about the sample. The wearable electronic device may also sequentially photograph a plurality of individual organic samples applied to different immunoassay regions of a test strip.

### CROSS-REFERENCE TO RELATED APPLICATIONS

The present application claims the priority benefit of U.S. provisional patent application No. 62/110,967, filed Feb. 2, 2015 and entitled “WEARABLE WITH TEST STRIPS AND OPTICAL READER FOR SKIN,” the disclosure of which is hereby incorporated by reference.

### BACKGROUND

### Field of the Invention

The present invention generally relates to a wearable device that analyzes a sample using an optical sensor. More specifically, the wearable device analyzes a sample embedded in or located on a replaceable immunoassay region.

### Description of the Related Art

Wearable technology may include any type of mobile electronic device that can be worn on the body, or attached to or embedded in clothes and accessories of an individual, and currently exists in the consumer marketplace. Processors and sensors associated with the wearable technology can display, process, and/or gather information. Such wearable technology has been used in a variety of areas, including monitoring health data of the user as well as other types of data and statistics. These types of devices may be readily available to the public and may be easily purchased by consumers. Examples of some wearable technology in the health arena include the Fitbit, Nike+ FuelBand, and Apple Watch.

Presently available electronic sensors coupled to a computing device are used to collect and manipulate data sensed by electronic sensors that are in contact with, or that communicate data over, the skin of a person. Immunoassay paper is a paper designed to characterize samples such as organic matter from a person. Examples of organic matter include, yet are not limited to the sweat, saliva, and blood from a person. Today, the pH or acid/base composition of an organic sample may be characterized by observing the color of an organic sample that has been embedded in or on a piece of immunoassay paper. The color, hue, or other characteristic of an organic sample on immunoassay paper is known to vary with the pH or acid/base composition of the sample. An individual may determine the pH or acid/base composition of an organic sample by viewing or photographing the sample after it has contacted one or more pieces of immunoassay paper. Current methods for viewing or analyzing organic samples on a piece of immunoassay paper are limited to viewing or photographing a single sample on a single piece of immunoassay paper.

Accordingly, there is a continued need for a wearable electronic device capable of quickly analyzing a plurality of organic samples.

### SUMMARY OF THE PRESENTLY CLAIMED INVENTION

The present invention generally relates to a wearable electronic device that collects an image of test sample using an optical sensor. The wearable device may photograph and analyze one or more organic samples embedded in or located on one or more immunoassay regions of a test strip. According to an embodiment, the device may sequentially photograph a plurality of individual organic samples embedded in or placed on one or more different immunoassay regions of a test strip that includes a plurality of immunoassay regions.

According to an embodiment, an apparatus of the present invention may include a plurality of electromagnets where each of the electromagnets generates a magnetic field when an electrical current is passed through or when power is applied to the electromagnets. An apparatus of the present invention may also include a memory, a processor, and an optical reader.

According to an embodiment is a test strip that includes a plurality of immunoassay regions and a plurality of magnets may be placed into or on an apparatus of the present invention. When power is applied to the plurality of electromagnets, magnetic fields generated by the electromagnets interact with magnetic fields from the magnets in the test strip. The interacting magnetic fields may cause the test strip to move from a first position to a second position.

An optical image or a photograph of a first immunoassay region on the test strip may be acquired using the optical sensor when the test strip is in the first position. After the test strip has been moved to a second position by the interacting magnetic fields, an optical image of a second immunoassay region on the test strip may be acquired using the optical sensor. The first and second optical images may be stored in the memory and a processor executing instructions out of the memory may identify a color, hue, or other characteristic of the first and/or the second immunoassay regions. The processor may also generate a result that identifies a health condition that corresponds to the color, hue, or other characteristic of either the first or the second immunoassay region.

According to an embodiment, a wearable sample analysis device includes: (i) a chamber configured to removably receive at least a portion of a movable test strip with a plurality of spaced immunoassay regions; (ii) an opening configured to provide access of a sample to one of the immunoassay regions; (iii) an optical reader component configured to move the test strip from a first position to a second position, each of the first and second positions aligning one of the immunoassay regions with the opening to receive a sample; (iv) an optical reader configured to acquire an optical image of a first immunoassay region when the test strip is in the first position, and further configured to acquire an optical image of a second immunoassay region when the test strip is in the second position; and (v) a processor in communication with the optical reader and configured to analyze at least one of the first and second optical images. The test strip is then removed from the chamber after each of the plurality of immunoassay regions receives a sample via the opening.

According to an embodiment, the movable test strip is rotatable within the chamber.

According to an embodiment, the processor is further configured to identify at least one color characteristic of the optical image of the first immunoassay region.

According to an embodiment, the opening is configured to receive a sample from the skin of a person.

According to an embodiment, the first immunoassay region is configured to receive a sample from a body of a person.

According to an embodiment, the sample is a skin sample, a saliva sample, a sweat sample, a blood sample, or a urine sample, among other types of samples.

According to an embodiment, the test strip includes a plurality of spaced magnets, and the optical reader component includes a plurality of electromagnets such that an electrical current flowing through the plurality of electromagnets generates a magnetic field that interacts with one or more of the plurality of spaced test strip magnets to move the test strip from the first position to the second position.

According to an embodiment, a method for analyzing a plurality of samples includes the steps of: (i) providing a wearable sample analysis device comprising a chamber configured to removably receive at least a portion of a movable test strip with a plurality of spaced immunoassay regions, an optical reader component configured to move the test strip from a first position to a second position, an optical reader configured to acquire an optical image, and a processor; (ii) acquiring, with the optical reader, an optical image of the first immunoassay region of the test strip in the first position; and (iii) analyzing, with the processor, the optical image to determine a characteristic of the first immunoassay region.

According to an embodiment, the method further includes the steps of moving, by the optical reader component, the test strip from the first position to the second position, and acquiring, with the optical reader, an optical image of the second immunoassay region of the test strip in the second position.

According to an embodiment is a non-transitory computer readable storage medium having embodied thereon a program executable by a processor to perform a method. The method includes the steps of: (i) providing, to a first immunoassay region of a test strip of a wearable sample device, a sample; (ii) acquiring, with an optical reader of the wearable sample device, an optical image of the first immunoassay region of the test strip; and (iii) analyzing, with a processor of the wearable sample device, the optical image to determine a characteristic of the first immunoassay region.

According to an embodiment, analyzing the first immunoassay region includes identifying at least one color that corresponds with a health condition.

According to an embodiment is a replaceable test strip for a wearable sample analysis device. The test strip includes a plurality of spaced immunoassay regions configured to receive a sample, the immunoassay regions configured to react to the sample to reveal a characteristic of the sample, wherein the replaceable test strip is configured to be moveable within a chamber of the wearable sample analysis device.

According to an embodiment, the test strip is circular and the plurality of spaced immunoassay regions are positioned around an outer region of the test strip.

According to an embodiment, the test strip comprises a plurality of spaced magnets extending outwardly from a plane of the test strip, the magnets positioned such that the polarity of each spaced magnet is opposite the polarity of a respective neighboring magnet. According to an embodiment, the plurality of spaced magnets are configured to interact with a plurality of magnets of the wearable sample analysis device, the interaction causing the test strip to rotate from a first position to a second position within the chamber.

According to an embodiment, the test strip is configured to be moved within the chamber by a motor of the wearable sample analysis device.

### BRIEF DESCRIPTION OF THE DRAWINGS

FIG. 1 illustrates an exemplary wearable user test strip device that may be used to analyze samples contained within or on a test strip, in accordance with an embodiment.

FIG. 2 illustrates a test strip carousel that may be placed in a wearable device optical reader, in accordance with an embodiment.

FIG. 3 illustrates the same test strip carousel and wearable device optical reader illustrated in FIG. 2, in accordance with an embodiment.

FIG. 4A illustrates a cross-section of the test strip carousel from position A to A′ and illustrates cross-section of the wearable device optical reader from position B to B′, in accordance with an embodiment.

FIG. 4B also illustrates cross-section of the test of carousel from position A to A′ and illustrates a cross-section of the wearable device optical reader from position B to B′, in accordance with an embodiment.

FIG. 5 illustrates a test strip being analyzed by an exemplary apparatus for analyzing samples, in accordance with an embodiment.

FIG. 6 illustrates a mobile device architecture that may be utilized to implement the various features and processes described herein, in accordance with an embodiment.

FIG. 7A illustrates a block diagram of an exemplary optical reader that may be used in a system for analyzing samples, in accordance with an embodiment.

FIG. 7B illustrates an exemplary optical reader apparatus that may be used in a system for analyzing samples, in accordance with an embodiment.

FIG. 8 illustrates an exemplary implementation of base software in an optical reader, in accordance with an embodiment.

FIG. 9 illustrates an optical reader software methodology that may be utilized to implement the various features and processes described herein, in accordance with an embodiment.

FIG. 10 illustrates an exemplary method where wearable test strips are read by an optical reader, in accordance with an embodiment.

### DETAILED DESCRIPTION

Embodiments of the present invention generally relate to a wearable electronic device that collects an image of test sample using an optical sensor. The wearable device may photograph and analyze one or more organic samples embedded in or located on one or more immunoassay regions of a test strip. An apparatus of the present invention may sequentially photograph a plurality of individual organic samples embedded in or placed on one or more different immunoassay regions of a test strip that includes a plurality of immunoassay regions.

An apparatus of the present invention may move or rotate a test strip that includes a plurality of immunoassay regions. When one or more organic samples are embedded in or placed on one or more immunoassay regions of the test strip, the pH or other characteristic of the organic samples may be analyzed after photographs of the different immunoassay regions have been acquired by an apparatus consistent with the present invention.

An individual using an apparatus consistent with the present invention may place a salvia sample on a first immunoassay region of a test strip. The individual may also place a sweat sample on a second immunoassay region of the test strip. An apparatus of the present invention may direct a user to put a test strip in a specific location in order to collect a sample. For example, the wearable device may display a message on a display instructing the user to place a test strip on their forehead to collect a sweat sample or to place a test strip in their urinary flow when collecting a urine sample. The message instructing the user may also be an audio message played over a speaker. After the samples have been placed on different immunoassay regions, the test strip may be placed in an apparatus of the present invention, and the apparatus may photograph and analyze each of the samples.

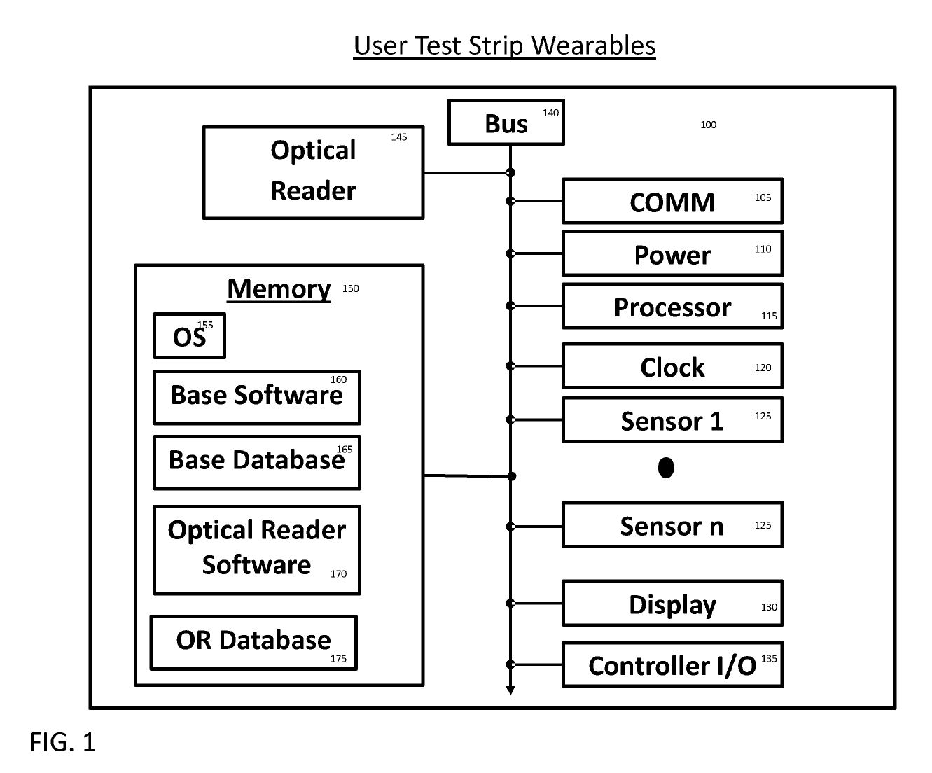

FIG. 1 illustrates an exemplary wearable user test strip device 100 that may be used to analyze one or more samples placed on a test strip. The user test strip wearable device 100 of FIG. 1 includes a communication interface 105, a power supply 110, a processor 115, a clock 120, internal sensors 1 through n 125, a display 130, and controller input output circuits (I/O) 135 communicating over at a bus 140. FIG. 1 also includes an optical reader 145 and a memory 150 connected to the communication bus 140. The memory 150 in FIG. 1 includes operating system software 155 (OS), base software 160, a base database 165, optical reader software 170, and an optical reading (OR) database 175.

During operation the wearable device 100 may display a message on display 130 instructing a user of the wearable device to place in the wearable device 100 a test strip that includes one or more immunoassay regions. After the user places a test strip in the wearable device 100, an immunoassay region in the test strip may be positioned over the optical reader 145 where an image of the immunoassay region is acquired by the optical reader 145. The optical reader 145 may also acquire an image of other immunoassay regions in the test strip, and the processor 115 may execute instructions from memory 150 when analyzing the acquired images. The analysis process may include comparing the acquired images with information from the OR database 175. When an acquired image is found to match an entry in the OR database, a message may be displayed on the display 130 that may inform the user of the wearable device 100 that a sample placed on the test strip appears to match a reference sample in the OR database 175. The message may include informing the user that the match appears to match or indicate a certain health condition.

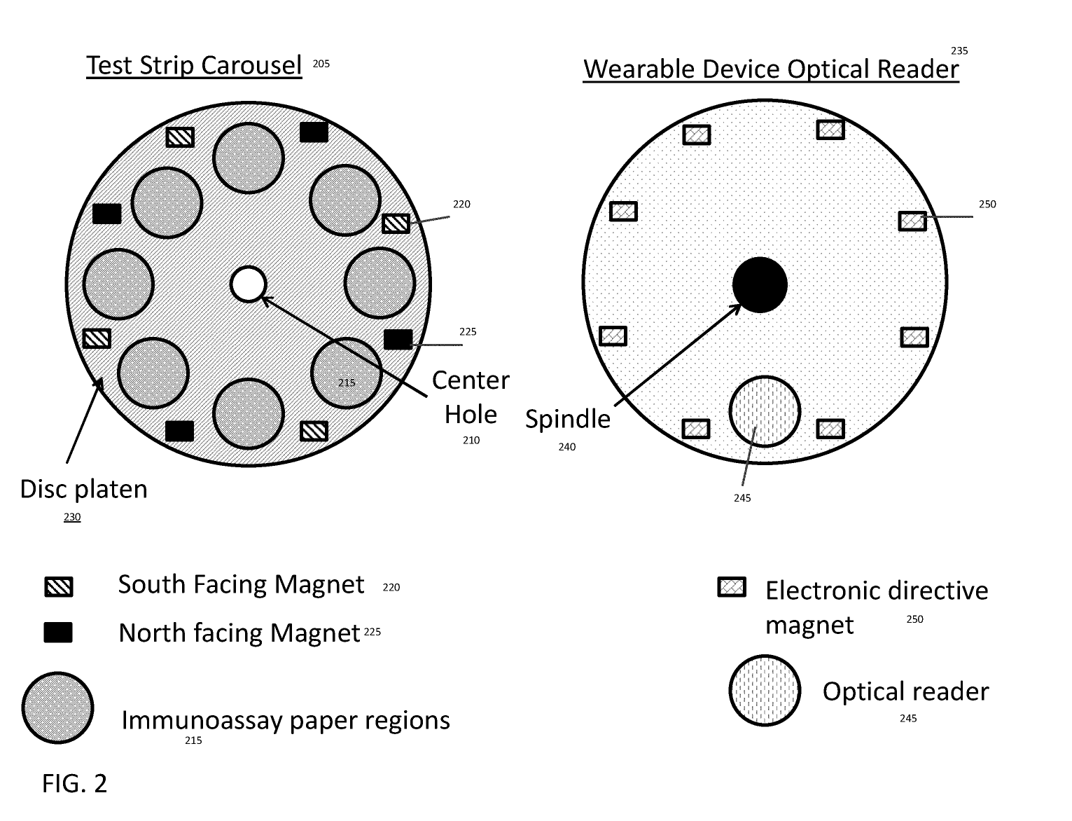

FIG. 2 illustrates a test strip carousel 205 that may be placed in or on a wearable device optical reader. The test strip carousel 205 may be any shape including, but not limited to, round, square, rectangular, and many other shapes. The test strip carousel 205 includes a center hole 210, a plurality of spaced immunoassay regions 215, a plurality of magnets 220 each with their South-seeking pole extending from the plane of the test strip carousel, and a plurality of magnets 225 each with their North-seeking pole extending from the plane of the test strip carousel. Each of the magnets 225 are depicted as solid black rectangular shapes, and each of the magnets 220 are depicted as rectangular boxes filled with slashes. As shown in FIG. 2, magnets 220 and 225 alternate around the outer region of test strip carousel 205. During use, one or more of the immunoassay regions 215 may comprise a sample that can be read, photographed, or otherwise analyzed by an optical reader 245. The test strip carousel 205 may be loaded with different types of immunoassay regions, and each type of immunoassay may be a specialized type of immunoassay. For example, specialized chemical immunoassay types may include an immunoassay region that can be used for testing urine, an immunoassay region that may be used for testing saliva, an immunoassay region that may be used to test sweat, or an immunoassay region that may be used to test other substances from a person, among many other possible immunoassay types.

According to an embodiment, the test strip carousel 205 is removably inserted into a chamber, slot, cavity, or other area or component of the wearable device 100. When the test strip carousel 205 is inserted or placed into the wearable device 100, one or more of the immunoassay regions 215 of the carousel are accessible for a sample to be placed on or in the immunoassay region. For example, the wearable device 100 can comprise a hole, slot, opening, wick, or other access point for a sample to be introduced to an immunoassay region of the carousel. Additionally, the immunoassay region will be accessible to the optical reader 145 of the wearable device 100 such that the optical reader can image or otherwise analyze the immunoassay region during and/or after introduction of the sample. The optical reader may obtain one or more images or other analyses of the immunoassay region during or after introduction of the sample. After an immunoassay region is utilized, the test strip carousel 205 is advanced such that a second of the plurality of immunoassay regions 215 is accessible via the access point formed in the wearable device 100. The process can be repeated until each of the plurality of immunoassay regions 215 are used, at which time the test strip carousel 205 can be removed from the chamber, slot, cavity, or other area or component of the wearable device 100 and a new test strip carousel 205 can be placed or inserted into the wearable device 100.

According to an embodiment, the wearable device 100 comprises an optical reader component 235 to obtain images or other analysis of the immunoassay regions 215 of the test strip carousel 205. Optical reader component 235 can also act as a stator of an electric motor to advance the carousel from one region to the next. The wearable device optical reader component 235 depicted in FIG. 2 includes a spindle 240, an optical reader 245, and a plurality of spaced electromagnetic drive magnets 250. The electromagnet drive magnets 250 may be constructed using a plurality of coils of wire (or conductive material), such that when an electrical current is passed through the coils of wire, magnetic fields surrounding the coils will be generated. By changing the electromagnetic orientation of the electromagnetic drive magnets, the wearable device optical reader may rotate the test strip carousel 205 when the test strip carousel 205 is present in the wearable device optical reader component 235. For example, alternating and oppositely-oriented magnetic fields of the spaced electromagnetic drive magnets 250 can interact with both the South facing magnets 220 and North facing magnets 225 in the test strip carousel 205. The electromagnetic drive magnets 250 cause the test strip carousel 205 to rotate when the magnetic fields in the electromagnetic drive magnets 250 are reversed. In other words, the electromagnetic drive magnets 250 interacting with the North 225 and South 220 facing magnets on the test strip carousel 205 reverse their orientation, causing the test strip carousel to rotate from one detent position to another such that a different immunoassay region located on the test strip 205 is oriented to the optical reader 245 for each detent position. By varying the magnetic fields of the electromagnetic drive magnets 250, the test strip carousel 205 may be rotated in a clockwise direction or in a counterclockwise direction. Each time the test strip carousel is rotated to a new position, a different immunoassay region is positioned relative to the optical reader 245.

Using interacting magnetic fields to move or rotate a test strip carousel has numerous advantages. For example, the device has no moveable parts other than the moving test strip carousel. Rather than a motor, gear, or other moving component, the magnetic field system requires only modification of an electric current to result in changes in the magnetic fields and movement of the test strip carousel. Not only does the magnetic field system reduce power consumption, which is one of the key factors of wearable technology, but it also reduces moving parts, thereby improving durability and extending the life of the device. In addition, manufacturing costs are significantly lowered.

The wearable device 100 is not limited to using a magnet system to move the test strip carousel 205 from one position to another position. Various other types of mechanisms may be used to move the test strip carousel 205. Other types of drive mechanisms that may be used to move the test strip carousel 205 to one or more different positions include, but are not limited to, a gear and tooth actuator, a plunger advancer, manual manipulation by the user, and other possible mechanisms. For example, wearable device 100 can include a motor that drives a gear that interacts with the test strip carousel 205 to move the carousel to a new position within the device. Alternatively, the user may be required to move the carousel by hand to a new position within the device.

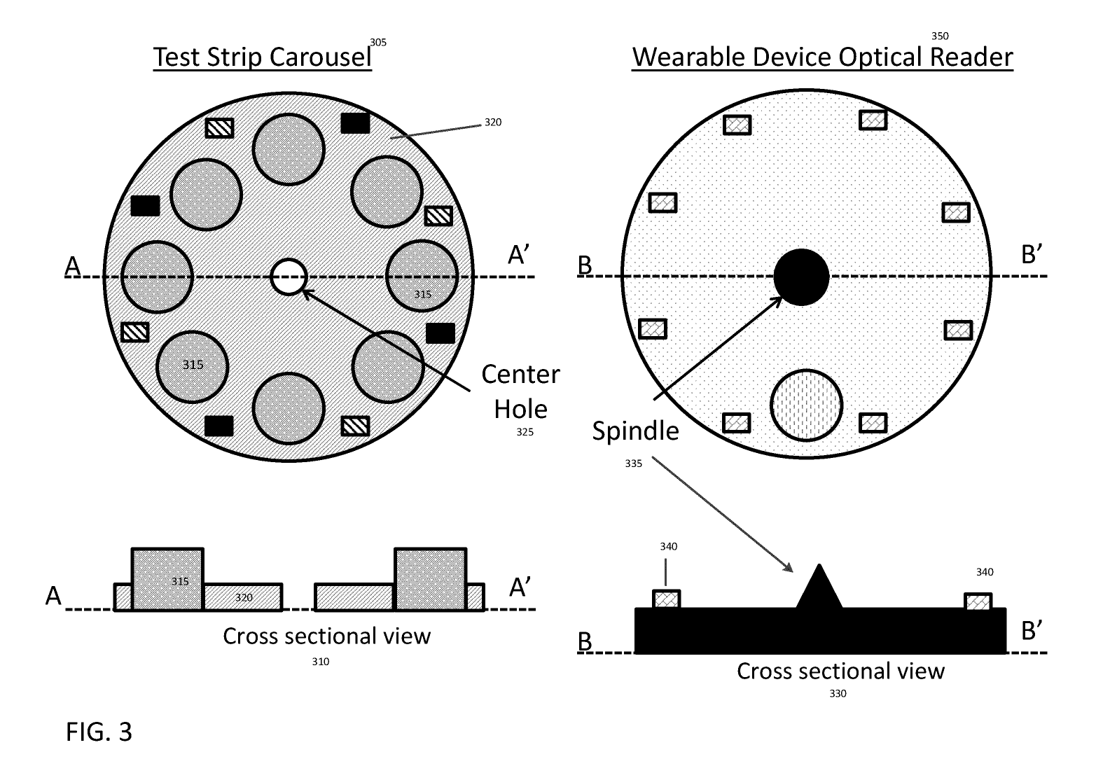

FIG. 3 illustrates the test strip carousel and wearable device optical reader illustrated in FIG. 2. FIG. 3 illustrates a side view of a cross-section 310 of test strip carousel 305 cut from position A to A′. Cross-section 310 of the test strip carousel 305 includes two areas where the test strip has a greater thickness 315. These areas of greater thickness are two of the plurality of immunoassay regions of the test strip carousel 305, each of which may receive a sample of organic matter, human tissue, or a human secretion, among other samples. The areas of test strip carousel in a test of carousel that do not comprise immunoassay test results in FIG. 3 are thinner regions 320. A center hole in the cross section 325 of the test strip carousel 305 is depicted as a void in the center of the cross section.

FIG. 3 also illustrates a side view of a cross-section 330 of the wearable device optical reader component 350 cut from position B to B′. The B to B′ cross-section 330 includes a large black region with a pointy portion 335 in the center of the cross-section. The pointy portion 335 in the B to B′ cross-section is a spindle 335 in the wearable device optical reader component 350. The B to B′ cross-section 330 of FIG. 3 also includes two electronic drive magnets 340 extending from the surface of the black region of the cross-section 330.

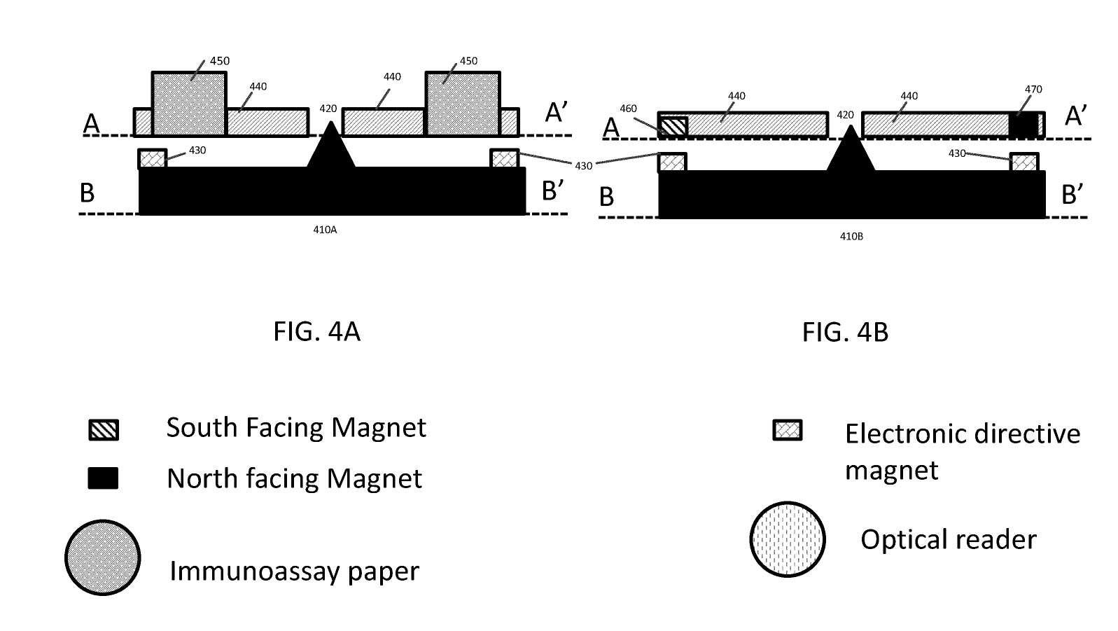

FIG. 4A illustrates a side view of a cross-section of the test strip carousel 305 from position A to A′ of FIG. 3, and illustrates a side view of a cross-section 410a of the wearable device optical reader component 350 from position B to B′ of FIG. 3. Here again the black region of the cross-section 410A of the wearable device optical reader includes a pointed spindle 420 that is aligned with the hole 210 in the test strip carousel A to A′ cross-section of FIG. 4A. The B to B′ cross-section of the wearable device optical reader also includes electromagnetic drive magnets 430 that are used to position the test strip carousel 440. FIG. 4A also illustrates the immunoassay regions 450 having a thicker cross-section as compared to the thinner regions of the test strip 440 which do not include an immunoassay region.

FIG. 4B illustrates a side view of a cross-section of a test strip carousel from position A to A′, and illustrates a side view of a cross-section of the wearable device optical reader component 350 from position B to B′ of FIG. 3. The cross-section A to A′ of test strip carousel 440 in FIG. 4B does not include an immunoassay region, as the test strip carousel depicted in FIG. 4B is between zones (or detents) where the immunoassay regions are located.

As depicted in FIG. 4B, the magnets of test strip carousel 440 are interacting with the magnets of the optical reader component. The cross-section A to A′ of test strip carousel 440 in FIG. 4B includes a South facing magnet 460 and a North facing magnet 470. The cross-section B to B′ of the wearable device optical reader component in FIG. 4B also includes magnetic drive magnets 430 which are positioned underneath the South facing magnet 460 and the North facing magnet 470 of test strip carousel 440. This positioning allows the magnetic field of magnets 460 and 470 of the test strip carousel to directly interact with the adjustable magnetic field of electromagnetic drive magnets 430 of the wearable device optical reader component.

According to an embodiment, the direction of drive current flowing through coils in electromagnetic drive magnets 430 may be changed in order to direct movement of the test strip carousel. Magnetic fields emanating from the electromagnetic drive magnets interact with magnetic fields from the South facing magnets 460 and the North facing magnets 470. Changing the direction of the current through the coils in the electromagnetic drive magnets 430 changes the polarity of magnetic fields emanating from the electromagnetic drive magnets 430, and the interaction with the magnetic fields of magnets 460 and 470 of the test strip carousel changes, causing the test strip carousel to move or otherwise change position or direction. For example, the magnetic fields from the electromagnetic drive magnets 430 can forcefully interact with the South facing magnets 460 and the North facing magnets 470 such that the test strip carousel is pushed or pulled in a first direction or in a second direction, depending on the polarity of the electromagnetic drive magnets 430.

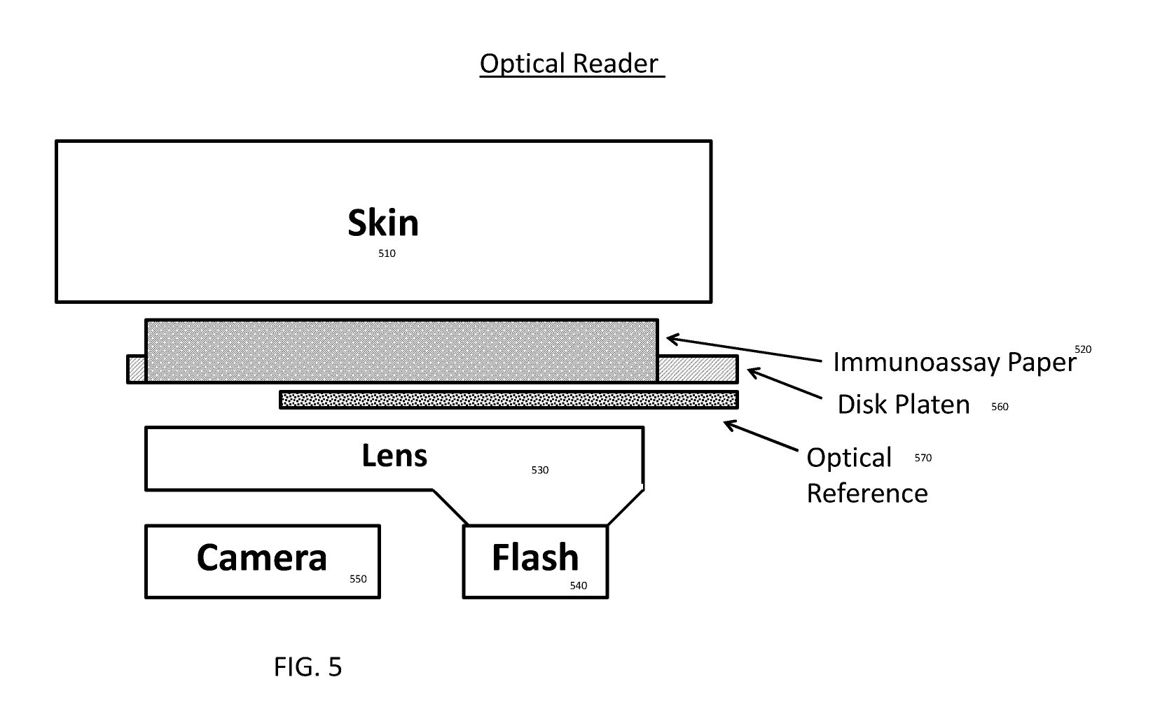

FIG. 5 illustrates an embodiment of a system configured to analyze one or more samples obtained from the skin of an organism. The system depicted in FIG. 5 includes a cross-section of an organism's skin 510 from which a sample is obtained, immunoassay region 520, a test strip carousel disc platen 560, an optical reference 570, a lens 530, a camera 550, and a flash 540. Many other configurations of the system depicted in FIG. 5 are possible.

When a sample has been applied to the immunoassay region 520, and the immunoassay region is positioned in front of the camera lens 530, a picture may be acquired of the immunoassay region 520. A color of the photograph of the immunoassay region may, for example, indicate health condition associated with the person or other organism from whom the sample was obtained. For example, when the immunoassay region 520 could receive a salivary sample from a person, and the color of the immunoassay region after receiving the salivary sample may correspond to the pH of the salivary sample. When the pH of the salivary sample corresponds to an acidic pH, the person may be at risk of losing enamel on their teeth. According to an embodiment the immunoassay region may be used in close proximity with the skin 510 of a person, and thus may easily receive a salivary sample or another type of sample obtained from the body of the person. The wearable device may direct a user to put a test strip in a specific location in order to collect a sample. For example, the wearable device may display a message on the display instructing the user to place the immunoassay region on their forehead to collect a sweat sample or to place the immunoassay region in their urinary flow to collect a urine sample. The message instructing the user may also be an audio message played over a speaker.

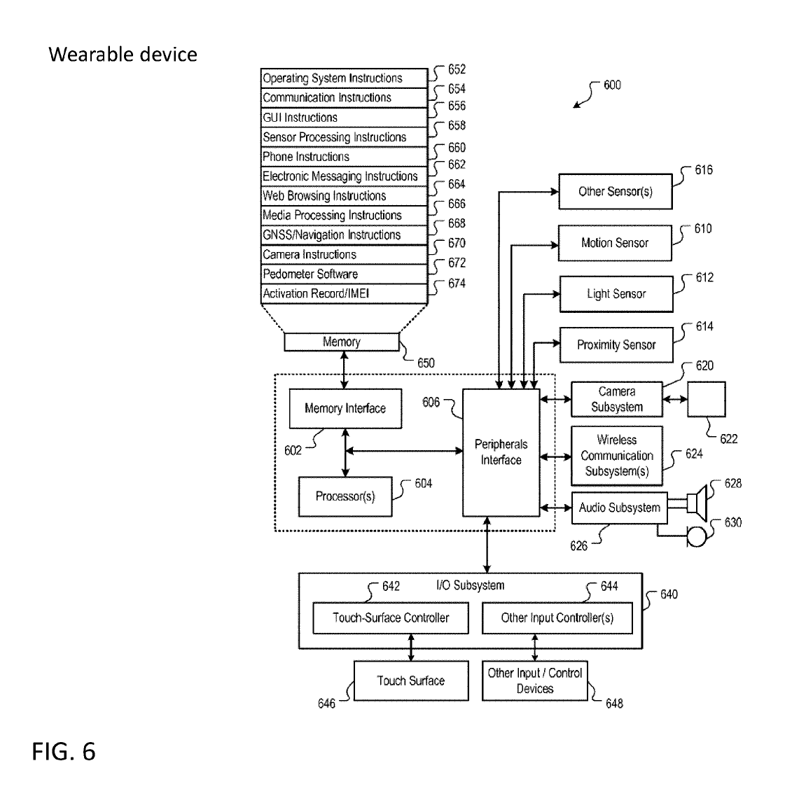

FIG. 6 illustrates a mobile device architecture that may be utilized to implement the various features and processes described herein. Architecture 600 can be implemented in any number of portable devices including but not limited to smart wearable devices. Architecture 600 as illustrated in FIG. 6 includes memory interface 602, processors 604, and peripheral interface 606. Memory interface 602, processors 604 and peripherals interface 606 can be separate components or can be integrated as a part of one or more integrated circuits. The various components can be coupled by one or more communication buses or signal lines.

Processors 604 as illustrated in FIG. 6 are meant to be inclusive of data processors, image processors, central processing unit, or any variety of multi-core processing devices. Any variety of sensors, external devices, and external subsystems can be coupled to peripherals interface 606 to facilitate any number of functionalities within the architecture 600 of the exemplar mobile device. For example, motion sensor 610, light sensor 612, and proximity sensor 614 can be coupled to peripherals interface 606 to facilitate orientation, lighting, and proximity functions of the mobile device. For example, light sensor 612 could be utilized to facilitate adjusting the brightness of touch surface 646. Motion sensor 610, which could be exemplified in the context of an accelerometer or gyroscope, could be utilized to detect movement and orientation of the mobile device. Display objects or media could then be presented according to a detected orientation (e.g., portrait or landscape).

Other sensors could be coupled to peripherals interface 606, such as a temperature sensor, a biometric sensor, or other sensing device to facilitate corresponding functionalities. Location processor 615 (e.g., a global positioning transceiver) can be coupled to peripherals interface 606 to allow for generation of geo-location data thereby facilitating geo-positioning. An electronic magnetometer 616 such as an integrated circuit chip could in turn be connected to peripherals interface 606 to provide data related to the direction of true magnetic North whereby the mobile device could enjoy compass or directional functionality. Camera subsystem 620 and an optical sensor 622 such as a charged coupled device (CCD) or a complementary metal-oxide semiconductor (CMOS) optical sensor can facilitate camera functions such as recording photographs and video clips.

Communication functionality can be facilitated through one or more communication subsystems 624, which may include one or more wireless communication subsystems. Wireless communication subsystems 624 can include 802.5 or Bluetooth transceivers as well as optical transceivers such as infrared. Wired communication system can include a port device such as a Universal Serial Bus (USB) port or some other wired port connection that can be used to establish a wired coupling to other computing devices such as network access devices, personal computers, printers, displays, or other processing devices capable of receiving or transmitting data. The specific design and implementation of communication subsystem 624 may depend on the communication network or medium over which the device is intended to operate. For example, a device may include wireless communication subsystem designed to operate over a global system for mobile communications (GSM) network, a GPRS network, an enhanced data GSM environment (EDGE) network, 802.5 communication networks, code division multiple access (CDMA) networks, or Bluetooth networks. Communication subsystem 624 may include hosting protocols such that the device may be configured as a base station for other wireless devices. Communication subsystems can also allow the device to synchronize with a host device using one or more protocols such as TCP/IP, HTTP, or UDP.

Audio subsystem 626 can be coupled to a speaker 628 and one or more microphones 630 to facilitate voice-enabled functions. These functions might include voice recognition, voice replication, or digital recording. Audio subsystem 626 in conjunction may also encompass traditional telephony functions.

I/O subsystem 640 may include touch controller 642 and/or other input controller(s) 644. Touch controller 642 can be coupled to a touch surface 646. Touch surface 646 and touch controller 642 may detect contact and movement or break thereof using any of a number of touch sensitivity technologies, including but not limited to capacitive, resistive, infrared, or surface acoustic wave technologies. Other proximity sensor arrays or elements for determining one or more points of contact with touch surface 646 may likewise be utilized. In one implementation, touch surface 646 can display virtual or soft buttons and a virtual keyboard, which can be used as an input/output device by the user.

Other input controllers 644 can be coupled to other input/control devices 648 such as one or more buttons, rocker switches, thumb-wheels, infrared ports, USB ports, and/or a pointer device such as a stylus. The one or more buttons (not shown) can include an up/down button for volume control of speaker 628 and/or microphone 630. In some implementations, device 600 can include the functionality of an audio and/or video playback or recording device and may include a pin connector for tethering to other devices.

Memory interface 602 can be coupled to memory 650. Memory 650 can include high-speed random access memory or non-volatile memory such as magnetic disk storage devices, optical storage devices, or flash memory. Memory 650 can store operating system 652, such as Darwin, RTXC, LINUX, UNIX, OS X, ANDROID, WINDOWS, or an embedded operating system such as VXWorks. Operating system 652 may include instructions for handling basic system services and for performing hardware dependent tasks. In some implementations, operating system 652 can include a kernel.

Memory 650 may also store communication instructions 654 to facilitate communicating with other mobile computing devices or servers. Communication instructions 654 can also be used to select an operational mode or communication medium for use by the device based on a geographic location, which could be obtained by the GPS/Navigation instructions 668. Memory 650 may include graphical user interface instructions 656 to facilitate graphic user interface processing such as the generation of an interface; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GPS/Navigation instructions 668 to facilitate GPS and navigation-related processes, camera instructions 670 to facilitate camera-related processes and functions; and instructions 672 for any other application that may be operating on or in conjunction with the mobile computing device. Memory 650 may also store other software instructions for facilitating other processes, features and applications, such as applications related to navigation, social networking, location-based services or map displays.

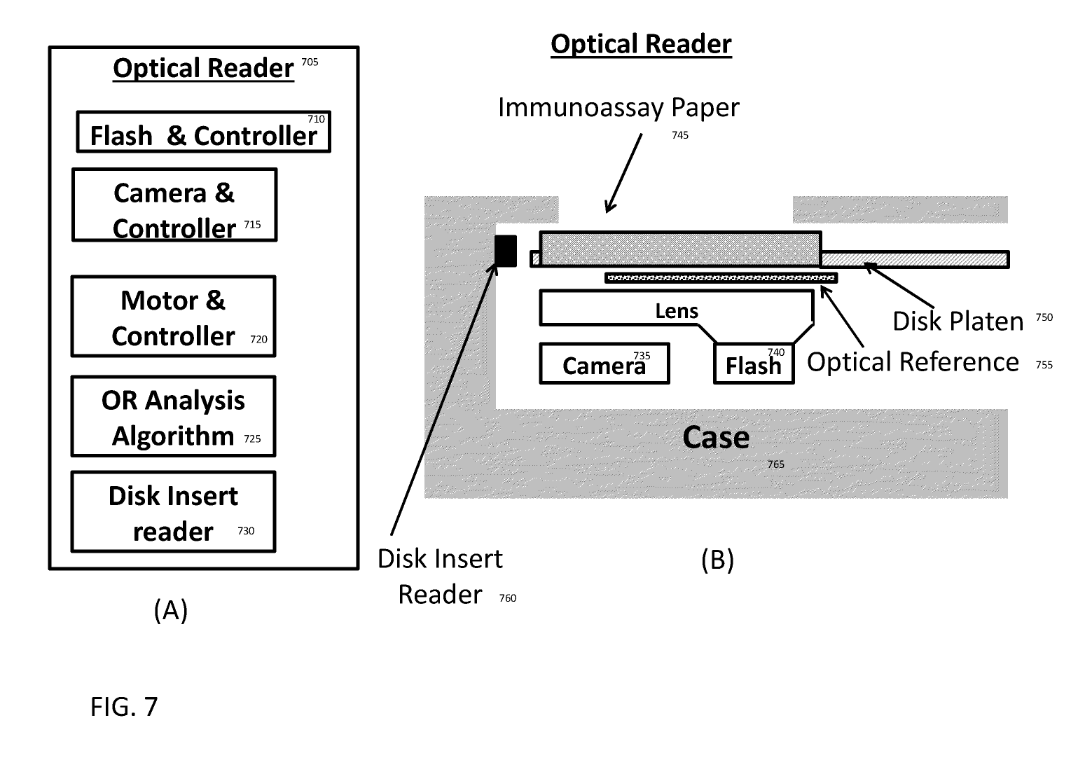

FIG. 7A illustrates a block diagram of an exemplary optical reader 705 in accordance with an embodiment of the present invention. The optical reader 705 in FIG. 7 includes a flash and a flash controller 710, a camera and a camera controller 715, a motor and a motor controller 720, an optical reading (OR) analysis algorithm 725, and a disk insert reader 730. According to an embodiment, the motor controller 720 directs a motor of the device to rotate or otherwise position the test strip carousel 205 or 305 into the proper position, where a sample can be applied to an immunoassay region of the test strip carousel. After the motor and motor controller properly align the immunoassay region of the test strip carousel and a sample is applied, the camera may be used to take a photograph or image of the immunoassay region, and the OR analysis algorithm may then analyze the image to determine whether a color, hue, or other characteristic of the photograph is indicative of one or more health conditions.

FIG. 7B illustrates a cross-section side view of an exemplary wearable optical reader apparatus in accordance with an embodiment of the present invention. The optical reader apparatus comprises a case 765 which can include a camera 735, a flash 740, a lens 770, an optical reference 755, and one or more immunoassay regions 745 on a disk platen 750. For example, the disk platen 750 and immunoassay region 745 may comprise a test strip carousel 205 or 305. By controlling the distance between the immunoassay region 745 and the camera lens 770, and by controlling the flash 740 timing/intensity, the color, hue, or another characteristic of the immunoassay region 745 may be sampled and analyzed after applying a sample. According to an embodiment, a disk insert reader 760 may be or include a sensor that detects the presence of a disk platen 750 in the case 765. As shown in FIG. 7B, the disk platen 750 is removable from the case 765 of the wearable device. As a result, an old disk platen can be removed when all of the immunoassay regions are utilized, and a new disk platen with unused immunoassay regions can be inserted into the wearable device.

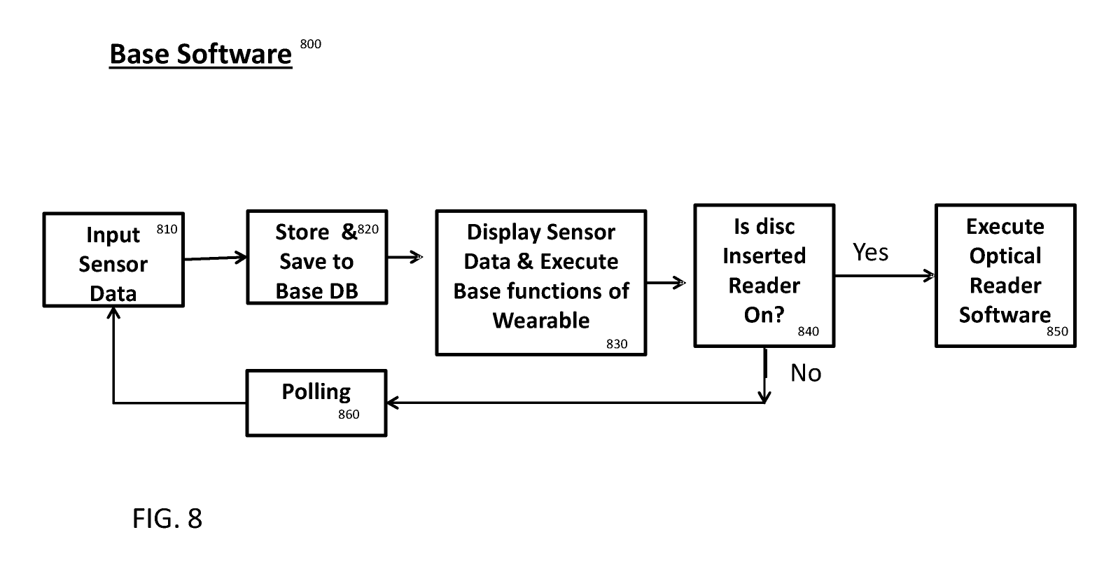

FIG. 8 illustrates an exemplary implementation of base software 800 in an optical reader device. The base software 800 of FIG. 8 begins with step 810, where sensor data is input from an optical sensor. In step 820, the sensor data is stored in a database.

Then in step 830 of FIG. 8, sensor data may be displayed on a display of the wearable device optical reader. This step 830 may also execute other software routines or implement certain base functions of the wearable device optical reader. The next step 840 of the base software flowchart is a determination step in which the system determines whether a disc is inserted in the reader, determines whether the disc reader is on, and determines whether there has been a trigger to obtain a test or obtain an image of an immunoassay region. When either of these is not true, program flow continues polling for input censor data 860.

In step 840, however, the system may determine that a disc is inserted in the reader, that the disc reader is on, and that there has been a trigger to obtain a test or obtain an image of an immunoassay region. A trigger that triggers a test may consist of any number of events. For example, a trigger may be based on a timer, where a trigger is automatically generated every 3 hours or some other amount of time. A trigger may also be based on sensor data. For example, when a sleep sensor senses that a person is sleeping, there may be a trigger to obtain a test or image. In yet another example, a sensor sensing that a pulse rate has increased over a threshold may be a trigger. In yet another instance, sensor data from a plurality of sensors may be combined, such as data from a pulse sensor and a calorie sensor. For example, when data obtained by the pulse sensor indicates that a person's heart rate is high and data obtained from the calorie sensor is high, such data may indicate that the person is exercising and this indication itself may trigger a test or image. Such a system may be used to run laboratory controlled experiments where data is acquired and analyzed when certain trigger events are detected. In certain instances, a trigger event may be according to a set of rules set by a doctor.

If the system has been triggered to obtain a test or image, the software may then move to step 850, where the optical reader software is executed. The optical reader software may, for example, direct the camera to obtain a photograph of a sample contained on the immunoassay region, and may direct the system to perform an analysis of the photograph.

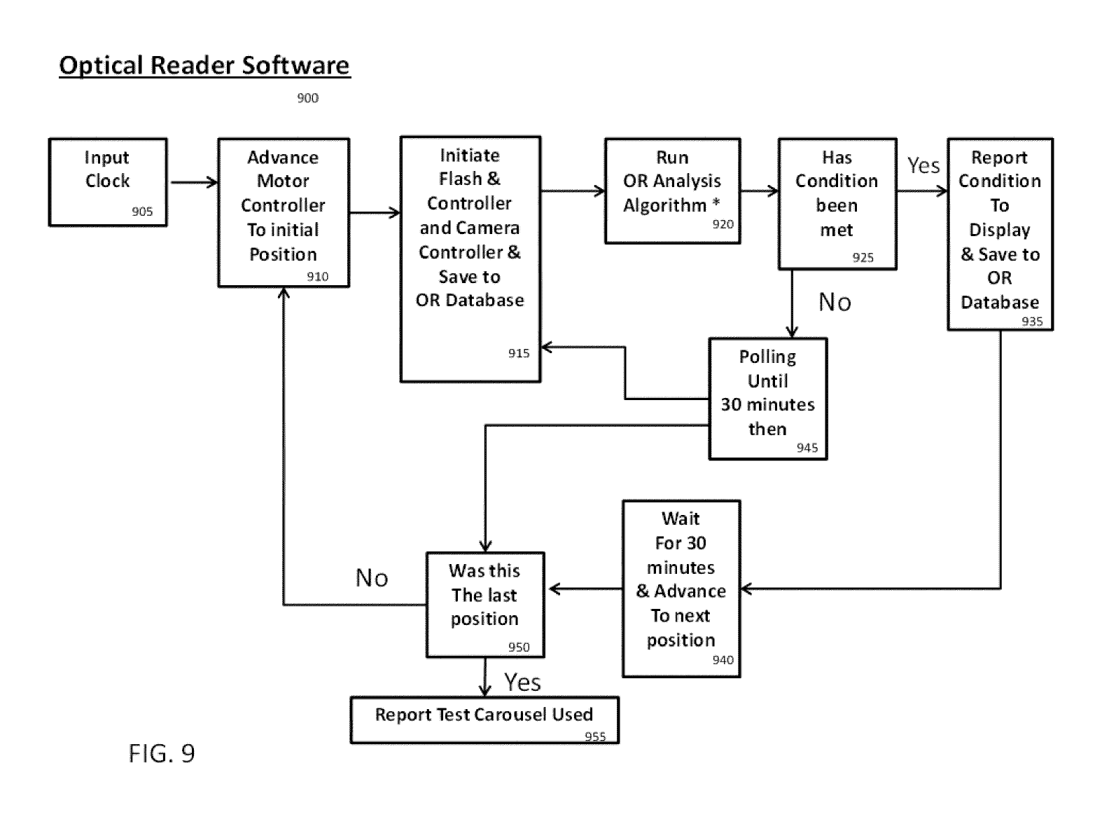

FIG. 9 illustrates an embodiment of optical reader software methodology that may be utilized to implement the various features and processes described herein. In step 905 of FIG. 9, the optical reader software 900 receives data from an input clock indicating that a sample has been triggered. Although step 905 indicates that the sample analysis trigger is a time-based trigger, many other triggers are possible as discussed herein. Then in step 910 of FIG. 9, a motor controller of the system advances a test strip carousel to an initial position. The initial position may correspond to an optical reader being located in proximity to an immunoassay region onto which a sample has been or can be applied or received. In step 915 of the method of FIG. 9, a photograph or other image of the immunoassay region is obtained by a camera. In certain instances, the photograph may be taken with a predetermined flash duration and intensity. Step 915 may also include saving the image data in an optical recognition (OR) database.

In step 920 of FIG. 9, an optical recognition algorithm may be run on a processor to analyze the collected image data. Then in step 925, the optical reader software may determine whether an operational condition has been met. If the operational condition has not been met, the program flow moves to step 945 where various inputs of the optical reader software may be polled for duration of time. For example, the system may wait a predetermined amount of time before obtaining another sample or taking another photograph. After step 945, another picture may be taken in step 915 of the method. Alternatively, the method may proceed to determination step 950, in which the system can determine whether the last immunoassay region of a test strip carousel has been photographed. If not, the method may return to step 910 and additional samples may be analyzed. If the last immunoassay region of a test strip carousel has been photographed, the method may proceed to step 955 where an indication that all of the immunoassay regions of the test strip carousel have been photographed (i.e. the test strip is used) may be presented to a user of the optical reader. The user can then remove the used test strip carousel, and can insert a new test strip carousel.

However, if the system determines in step 925 of the method that an operational condition has been met, the program flow moves to step 935 where a condition or a result of the analysis may be reported to a user, including but not limited to displaying on a display. The condition or result may also be saved in the OR database. Then, in step 940 of the method, the optical reader software may pause for a predetermined period of time, or before receiving another trigger, before then advancing the test strip carousel to a next position. After step 940, program flow moves to the determination step 950 described above.

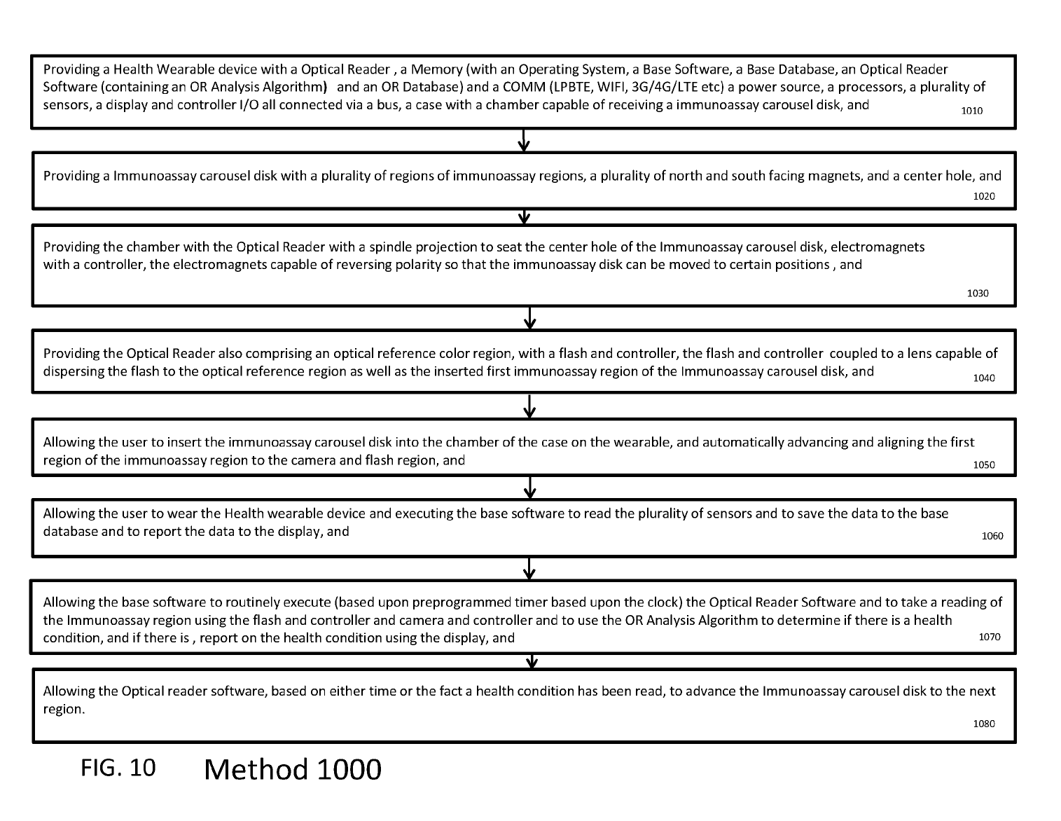

FIG. 10 illustrates an embodiment of a method for analyzing a sample utilizing a wearable device in which test strips are read by an optical reader. In step 1010 of the method depicted in FIG. 10, a wearable device may be provided with an optical reader. The wearable device may be any of the wearable devices described or otherwise envisioned herein. The wearable device may be, for example, provided with a memory that includes an operating system (OS) software, base software, a base database, optical reader software, and an OR database. In this step 1010, the wearable device or the optical reader may also be provided with a communication interface, a power source, a processor, a plurality of sensors, a display, and control input output (I/O) connections, among other components. The optical reader may also be provided with a chamber capable of removably receiving an immunoassay test strip carousel.

In step 1020 of the method depicted in FIG. 10, an immunoassay carousel is provided with a plurality of immunoassay regions. The immunoassay test strip carousel may also be provided with a plurality of North and South facing magnets and a center hole, as described herein.

In step 1030 of the method, an optical reader is provided with a chamber, with a spindle configured to receive an immunoassay carousel disc, and with a plurality of electromagnets.

In step 1040 of the method, the optical reader is provided with an optical reference color region, a flash, a controller, and a lens coupled to the flash. The lens may be capable of dispersing the flash when taking photographs of the immunoassay test strip carousel.

In step 1050 of the method depicted in FIG. 10, the immunoassay test strip carousel is inserted into a chamber of the optical reader. Once inside the chamber the immunoassay carousel disc may be advanced and aligned such that a first immunoassay region can receive a sample and/or is aligned with the lens of the camera.

Next, in step 1060, a user wears the wearable device of the present invention. The wearable device can execute base software and the base software may read data from a plurality of sensors. The base software may also store sensed data in a database or may display the data on a display at the health wearable device.

In step 1070 of the method of FIG. 10, the base software of the wearable device may execute one or more functions according the present invention. For example, a processor connected to the optical reader may be allowed to run software from a memory to acquire and analyze data from the one or more sensors. In certain instances the present invention may obtain a photograph or other reading from one or more immunoassay regions of the immunoassay test strip carousel. According to an embodiment, the optical reader may use flash photography, and the processor connected to the optical reader may use optical recognition analysis algorithms when determining whether a person has a health condition.

In step 1080 of the method of FIG. 10, the optical reader software may advance the immunoassay test strip carousel to a second or next region to receive a sample and be photographed and analyzed. By photographing and analyzing the color, hue, or other characteristic of a sample placed on or in an immunoassay region, the present invention may identify a health condition of the user of the wearable device. Samples that may be included in an immunoassay region include, but are not limited to, a skin sample, a saliva sample, a sweat sample, a blood sample, and a urine sample from a person, among many others.

The various methods may be performed by software operating in conjunction with hardware. For example, instructions executed by a processor, the instructions otherwise stored in a non-transitory computer readable medium such as memory. Various interfaces may be implemented—both communications and interface. One skilled in the art will appreciate the various requisite components of a mobile device and integration of the same with one or more of the foregoing figures and/or descriptions.

While various embodiments have been described above, it should be understood that they have been presented by way of example only, and not limitation. The descriptions are not intended to limit the scope of the invention to the particular forms set forth herein. Thus, the breadth and scope of a preferred embodiment should not be limited by any of the above-described exemplary embodiments. It should be understood that the above description is illustrative and not restrictive. To the contrary, the present descriptions are intended to cover such alternatives, modifications, and equivalents as may be included within the spirit and scope of the invention as defined by the appended claims and otherwise appreciated by one of ordinary skill in the art. The scope of the invention should, therefore, be determined not with reference to the above description, but instead should be determined with reference to the appended claims along with their full scope of equivalents.

1. A wearable sample analysis device, the apparatus comprising:
a chamber comprising at least a portion of a rotatable test strip, wherein the test strip comprises a plurality of spaced immunoassay regions;
an opening in the wearable device, wherein the opening receives a sample from the skin of a person, and wherein the sample accesses one of the plurality of spaced immunoassay regions;
a rotatable carousel, where in the rotatable carousel moves the rotatable test strip from a first position to a second position, wherein in each of said first and second positions a respective one of the plurality of immunoassay regions is aligned with said opening to receive a sample;
an optical reader, wherein the optical reader acquires an optical image of a first immunoassay region of the plurality of immunoassay regions of the test strip when the test strip is in the first position, and further acquires an optical image of a second immunoassay region of the plurality of immunoassay regions of the test strip when the test strip is in the second position; and
a processor in communication with the optical reader, wherein the processor analyzes at least one of the first and second optical images;
wherein the test strip is removed from the chamber after each of the plurality of immunoassay regions have received a sample via the opening.

2. (canceled)

3. The wearable sample analysis device of claim 1, wherein the processor identifies at least one color characteristic of the optical image of the first immunoassay region.

4. (canceled)

5. (canceled)

6. (canceled)

7. The wearable sample analysis device of claim 1, wherein the test strip comprises a plurality of spaced magnets, and further wherein the optical reader component device comprises a plurality of electromagnets, wherein an electrical current flowing through the plurality of electromagnets generates a magnetic field that interacts with one or more of the plurality of spaced test strip magnets such that the test strip is moved from the first position to the second position by the interacting magnetic fields.

8. A method for analyzing a plurality of samples, the method comprising:
providing a wearable sample analysis device comprising: (i) a chamber comprising at least a portion of a rotatable test strip comprising a plurality of spaced immunoassay regions; (ii) an opening, wherein the opening receives a sample from the skin of a person, and wherein the sample accesses one of the plurality of spaced immunoassay regions; (iii) a rotatable carousel, wherein the rotatable carousel moves the rotatable test strip from a first position to a second position, wherein in each of said first and second positions a respective one of the plurality of immunoassay regions is aligned with an opening to receive a sample; (iv) an optical reader, wherein the optical reader acquires an optical image; and (v) a processor;
acquiring, with the optical reader, an optical image of the first immunoassay region of the test strip in the first position;
analyzing, with the processor, the optical image to determine a characteristic of the first immunoassay region;
moving, by the rotatable carousel, the test strip from the first position to the second position, and
acquiring, with the optical reader, an optical image of the second immunoassay region of the test strip in the second position.

9. (canceled)

10. (canceled)

11. The method of claim 8, wherein the sample is at least one of a skin sample, a saliva sample, a sweat sample, a blood sample, or a urine sample.

12. A non-transitory computer readable storage medium having embodied thereon a program executable by a processor to perform a method, the method comprising:
providing, to a first immunoassay region of a rotatable test strip of a wearable sample device, a sample, wherein the rotatable test strip comprises a plurality of spaced immunoassay regions;
acquiring, with an optical reader of the wearable sample device, an optical image of the first immunoassay region of the test strip;
analyzing, with a processor of the wearable sample device, the optical image to determine a characteristic of the first immunoassay region;
moving, via a rotatable carousel of the wearable device, the test strip from a first position to a second position; and
acquiring, with the optical reader, an optical image of the second immunoassay region of the test strip in the second position.

13. (canceled)

14. The non-transitory computer readable storage medium of claim 12, wherein analyzing the first immunoassay region includes identifying at least one color that corresponds with a health condition.

15. The non-transitory computer readable storage medium of claim 12, wherein the sample is at least one of a skin sample, a saliva sample, a sweat sample, a blood sample, or a urine sample.

16. A circular test strip for a wearable sample analysis device, the test strip comprising:
a plurality of spaced immunoassay regions positioned around an outer region of the strip, wherein the spaced immunoassay regions are positioned to receive a sample from a user's skin via an opening of the wearable sample analysis device, wherein the immunoassay region reacts to the sample to reveal a characteristic of the sample;
wherein the replaceable test strip engages a motor within the wearable sample analysis device to move within a chamber of the wearable sample analysis device.

17. (canceled)

18. The replaceable test strip of claim 16, wherein the test strip comprises a plurality of spaced magnets extending outwardly from a plane of the test strip, the magnets positioned such that the polarity of each spaced magnet is opposite the polarity of a respective neighboring magnet.

19. The replaceable test strip of claim 18, wherein the plurality of spaced magnets interact with a plurality of magnets of the wearable sample analysis device, the interaction causing the test strip to rotate from a first position to a second position within the chamber.

20. (canceled)
